(19) United States
(12) Patent Application Publication
(10) Pub. No.: US 2022/0273399 A1
(43) Pub. Date: Sep. 1, 2022

(54) SYSTEMS COMBINING THERAPEUTIC LASERS AND CURING LIGHTS

(71) Applicant: CAO Group, Inc., West Jordan, UT (US)

(72) Inventors: Zhaohui LIN, Salt Lake City, UT (US); Densen CAO, Sandy, UT (US)

(73) Assignee: CAO Group, Inc., West Jordan, UT (US)

(21) Appl. No.: 17/631,491

(22) PCT Filed: Jul. 29, 2020
(86) PCT No.: PCT/US20/44038

§ 371 Date: Jan. 30, 2022

Related U.S. Application Data
(60) Provisional application No. 62/879,926, filed on Jul. 29, 2019.

(51) Int. Cl. A61C 1/00; A61B 18/22; A61C 13/15

(52) U.S. Cl. CPC A61C 1/0046; A61B 18/22; A61C 19/004; A61N 2005/0606

(57) ABSTRACT

The use of multiple frequency laser modules (312) and a selection of emitter heads (304) allow for systems (300) which may be used for a wide variety of medical and dental procedures. Each system (300) has a plurality of emitter heads (304) to select from either a curing light, a cutting laser, or any other therapeutic energy emission all while utilizing the same laser module (312). Various structures of emitter heads are disclosed, as well as laser modules and connection strategies.

### CROSS-REFERENCES TO RELATED APPLICATIONS

This application claims priority as a non-provisional perfection of prior filed U.S. Application No. 62/879,898, filed Jul. 29, 2019, and incorporates the same herein by reference in its entirety.

### FIELD OF THE INVENTION

The present invention relates to the field of medical, dental, and industrial instruments and more particularly relates to systems that may alternately emit radiant energy as either a therapeutic laser or a curing light.

### BACKGROUND OF THE INVENTION

In the past decades, mankind has harnessed the power of radiant energy for a multitude of purposes, particularly in the medical and dental fields. A curing light is an essential tool for use of light activated materials in a variety of industries. Particularly, curing lights are a daily tool that practitioner uses in dentistry for curing composites, adhesives, and other materials. It is desirable for a curing light to have a high-power parallel beam, an adjustable beam size, no light degradation from light emitting positions between 10-20 mm and be compact with either corded or battery powered operation. Curing lights using LEDs as the light source are widely used in the industry today. Diode lasers can be an alternative light source for curing lights with the development of advanced diode lasers in different wavelength ranges. Diode lasers can also be used as light sources for therapeutic applications including surgery, pain management, healing, coagulation, etc. in dentistry and medicine.

Historically, a practitioner would need separate devices for different purposes (i.e., a laser for cutting or other therapeutic purposes and a curing light for material manipulation). The present invention combines these functions in a single apparatus. Having the same apparatus for both therapeutic laser and other radiant energy functions saves precious floorspace and allows the practitioner to utilize the same instrument during a procedure where both the cutting power of a laser and the more mild effects of, for example, a curing light might be needed.

### SUMMARY OF THE INVENTION

In view of the foregoing disadvantages inherent in the known types of therapeutic lasers and curing lights, an improved combination therapeutic laser and curing light may provide a system which operates as both essential tools. Such a combination should meet the following objectives: that it provide effective laser and effective curing light functionality, that activation of either function be simple, intuitive, and efficient, that the final unit not occupy much more floor or other operational space as a unit which accomplished one of the stated functions alone (a “stand-alone unit”), that it be reasonably affordable in comparison to stand-alone units, that it may simply and efficiently be assembled to minimize complexity and cost. As such, a new and improved radiant energy system may comprise a laser source combined with selectable emitter heads or tips which affect laser output from the laser source in order to accomplish these objectives.

The more important features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also, it is to be understood that the phraseology and terminology employed herein are for description and should not be regarded as limiting.

As such, those skilled in the art will appreciate that the conception, upon which this disclosure is based, may readily be utilized as a basis for the designing of other structures, methods, and systems for carrying out the several purposes of the present invention. It is important, therefore, that the claims be regarded as including such equivalent constructions as far as they do not depart from the spirit and scope of the present invention.

### BRIEF DESCRIPTION OF THE DRAWINGS

FIG. 1 is a schematic drawing of a combination therapeutic laser and curing light system, utilizing a curing light head, as one embodiment of the invention.

FIG. 2 is the schematic of FIG. 1, where the combination therapeutic laser and curing light system is utilizing a cutting laser head.

FIG. 3 is the schematic of FIG. 1, where the combination therapeutic laser and curing light system is utilizing a diffuse laser head.

FIG. 4 is a schematic drawing of a combination therapeutic laser and curing light system, utilizing a curing light head, as a second embodiment of the invention.

FIG. 5 is the schematic of FIG. 4, where the combination therapeutic laser and curing light system is utilizing a cutting laser head.

FIG. 6 is the schematic of FIG. 4, where the combination therapeutic laser and curing light system is utilizing a diffuse laser head.

FIG. 7 is a schematic depicting one embodiment of a combination therapeutic laser and curing light system with a curing light head.

FIG. 8 is the combination therapeutic laser and curing light system of FIG. 7, with an alternate curing light attachment head.

FIG. 9 is the combination therapeutic laser and curing light system of FIG. 7, with another alternate curing light attachment head.

FIG. 10 is the combination therapeutic laser and curing light system of FIG. 7, with a further alternate curing light attachment head.

FIG. 11 is the combination therapeutic laser and curing light system of FIG. 7, with a still further alternate curing light attachment head.

FIG. 12 is the combination therapeutic laser and curing light system of FIG. 7, with an embodiment of a therapeutic cutting laser head.

FIG. 13 is the combination therapeutic laser and curing light system of FIG. 7, with an embodiment of a therapeutic large-area laser head.

FIG. 14 is a schematic drawing of a desktop system utilizing an embodiment of the combination therapeutic laser and curing light system.

FIG. 15 is a schematic drawing of a diode laser module for use in the combination therapeutic laser and curing light system.

FIG. 16 is a schematic drawing depicting an alternate diode laser module for use in the combination therapeutic laser and curing light system.

FIG. 17 is a schematic drawing depicting one embodiment of battery attachment for use in the combination therapeutic laser and curing light system.

FIG. 18 is a schematic drawing depicting one embodiment of head attachment for use in the combination therapeutic laser and curing light system.

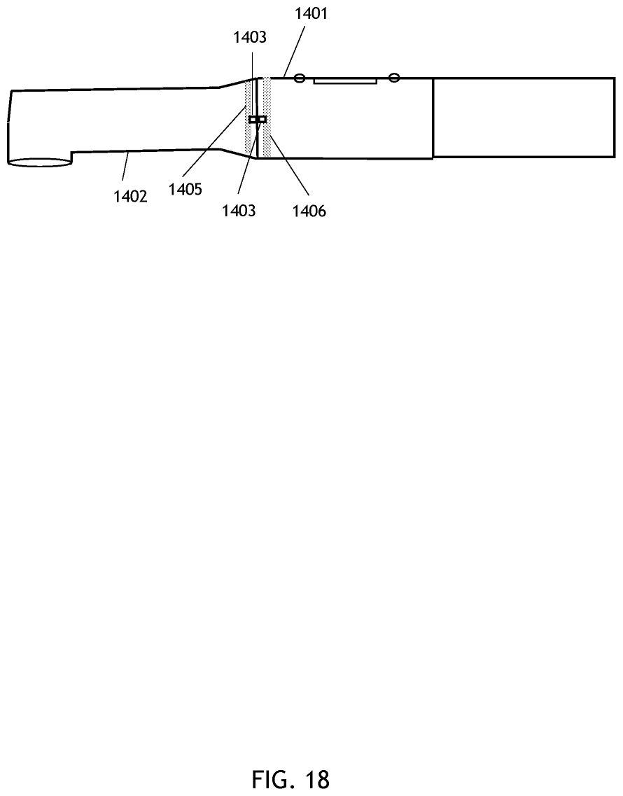

### DETAILED DESCRIPTION OF THE PREFERRED EMBODIMENT

With reference now to the drawings, multiple embodiments of the combination therapeutic laser and curing light system are herein described. It should be noted that the articles “a,” “an,” and “the,” as used in this specification, include plural referents unless the content clearly dictates otherwise.

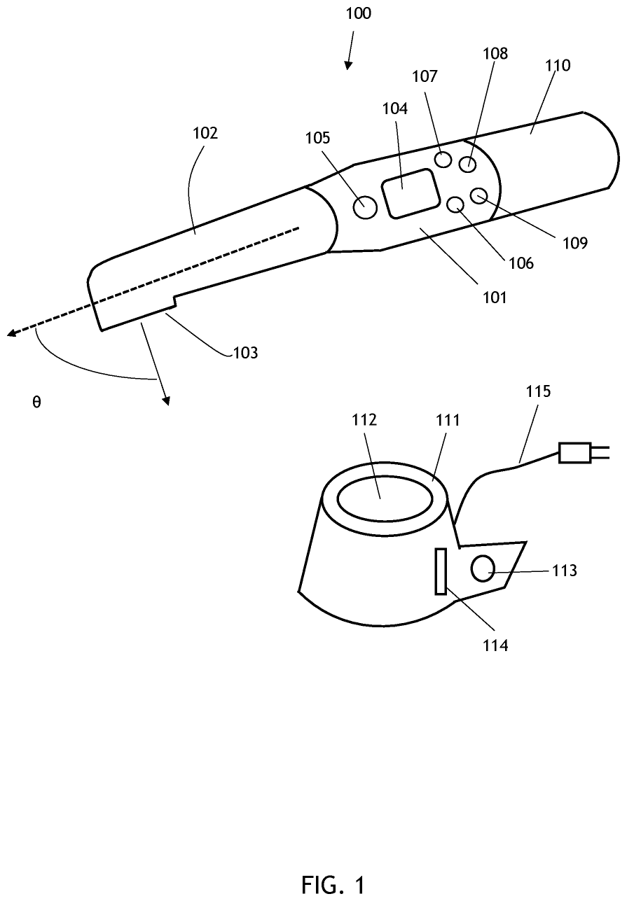

In FIG. 1, a curing light and laser system (100) features a main handpiece body (101) which can be made of metals, plastic, composite, or any other durable material. A curing light emitter head (102) includes a light exit (103). In some embodiments, the light exit (103) may be angled θ from 0 to 90 degrees in respect to a horizontal axis (dotted line in FIG. 1) of the curing head (102). The curing head (102) can be also be made of metal, plastic, composite, or any other durable materials. The curing head (102) can be rotated around the main body (101) and is removable therefrom. A display (104) may show the light operation status. This display (104) can be LCD, OLED, LED module, or any other type of display. Various selection buttons may be provided for light activation (105), a timer (106) and to adjust operation modes (107), which could include the activation of one of a plurality of laser emitting chips on a laser module, each with a unique frequency. A main power on/off switch (108) and an emergency stop switch (109) may be utilized to stop light operation. The light can be powered by either AC/DC power or battery. If AC/DC power is used, the power source can plug into the light directly. Alternately a rechargeable battery (110) can be attached or detached from the main body (101). A charging station (111) may be provided for the battery (110) whereby a space (112) for either the battery (110) or the main body (101) may be provided. The battery (110) can be charged through contacts or wireless induction charging or may charge by plugging a cord into the unit. System status, in particular potential light output intensity, may be measured in the charging station (111) as a light intensity window (114) and an indicator (113) may be provided. The charging station (111) may be powered by a cord with an AC wall plug (115).

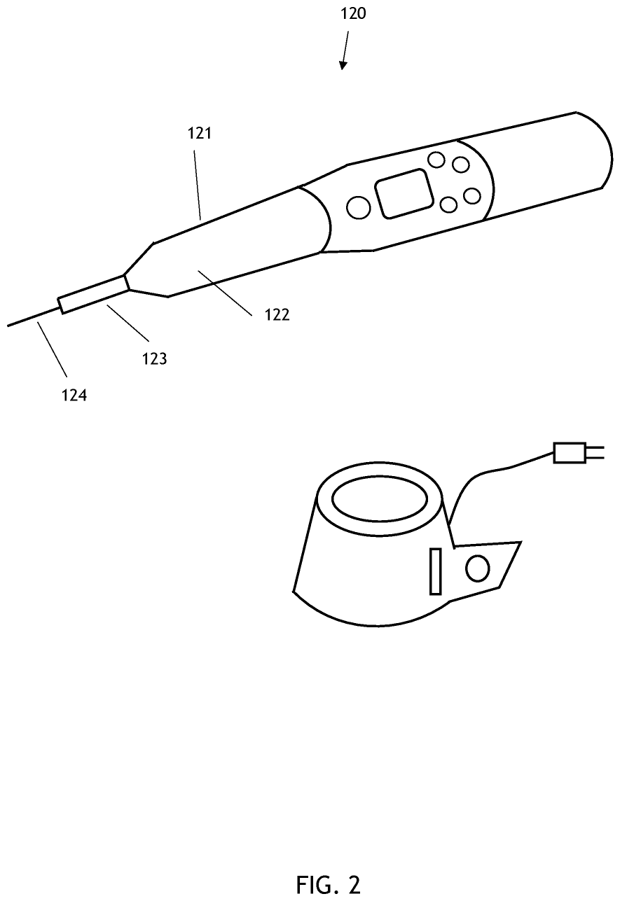

FIG. 2 shows a therapeutic laser system (120) which achieved by changing the emitter head on the system described in FIG. 1. The embodiments shown are the same except the head of the light is changed to one utilizing a therapeutic cutting head which is useful for various therapeutic purposes. The main body (122) of the head (121) can be made of metal, plastic, composite, or any other durable material. While one end of the head (121) is attached to main body of the system, the other features a cannular portion (123) from which an optical fiber (124) is extruded. The cannular portion (123) can be made of metal or plastic and may be bendable to any angle as desired. The fiber (124) can have size as small as 100 micrometers, but the entire head (121) may be configured to deliver different sizes or shapes of the beam.

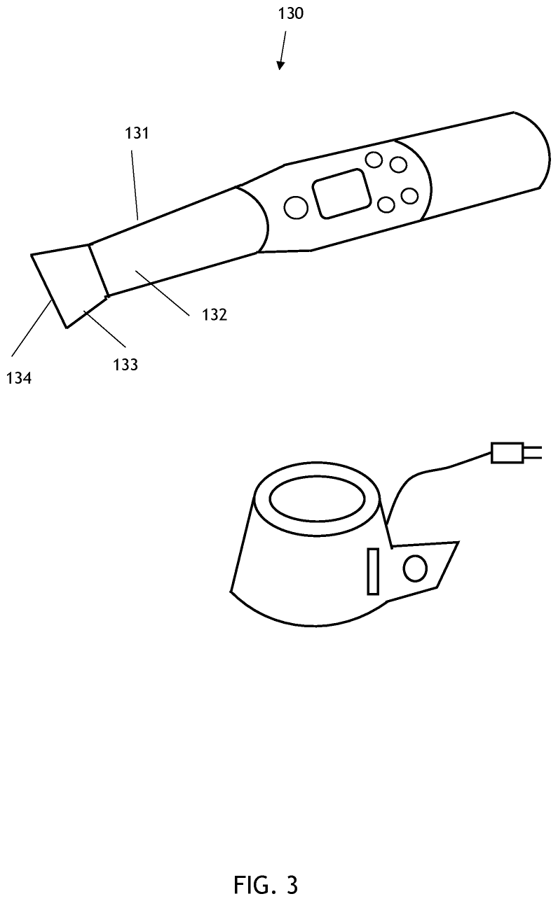

An alternate arrangement of the therapeutic system (130) may feature a head for therapy with large area of beam (FIG. 3). Like the system (120) shown in FIG. 2, this therapeutic laser system (130) can also be achieved by changing the emitter head of the system described in FIGS. 1 and 2. All the embodiments are the same except that the head of light changed to a different therapeutic head (131). In this embodiment a head for other therapeutic purposes (131) features a main body (132) which can be made of metals, plastic, composite, or any other durable materials. Light exits (134) the head at a cone (133) and its size and shape may be altered by the size and shape of the cone (133).

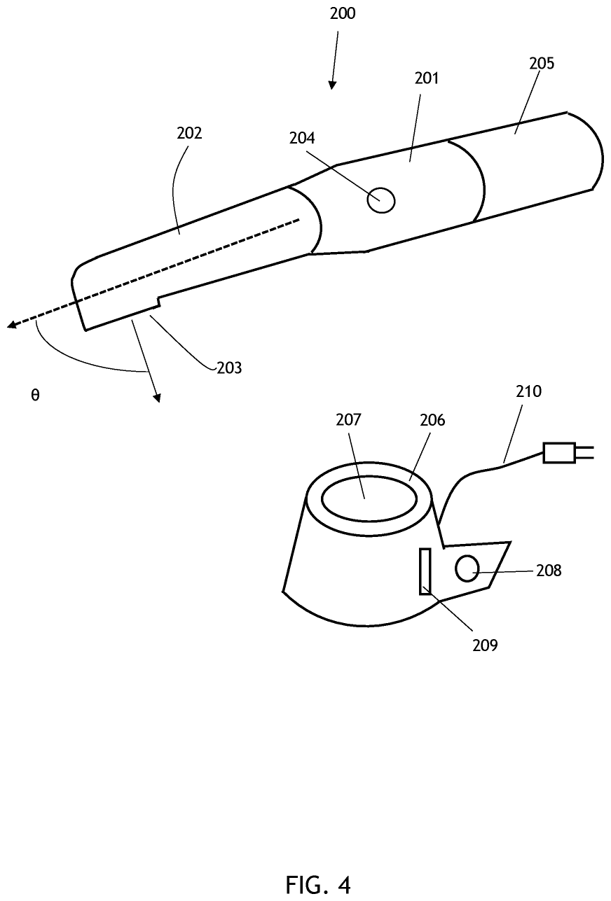

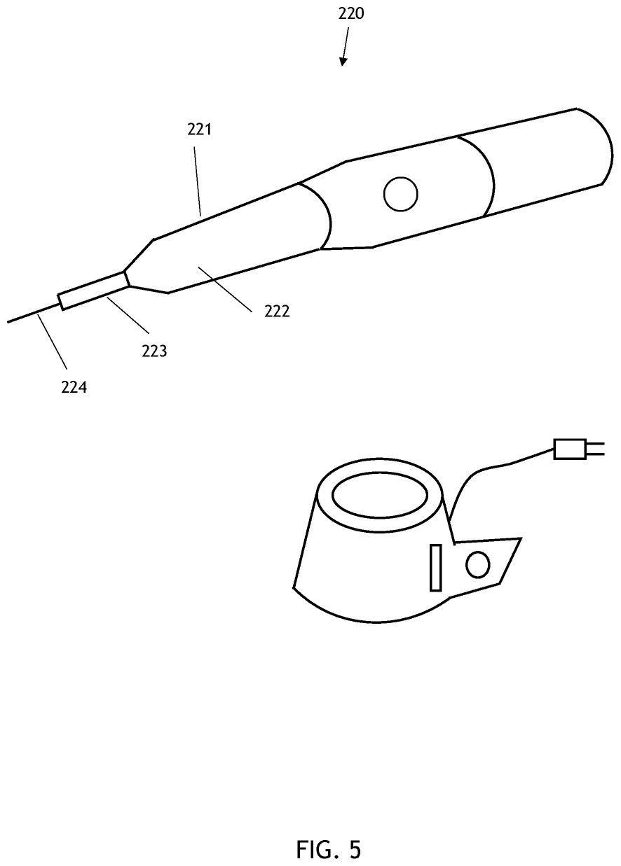

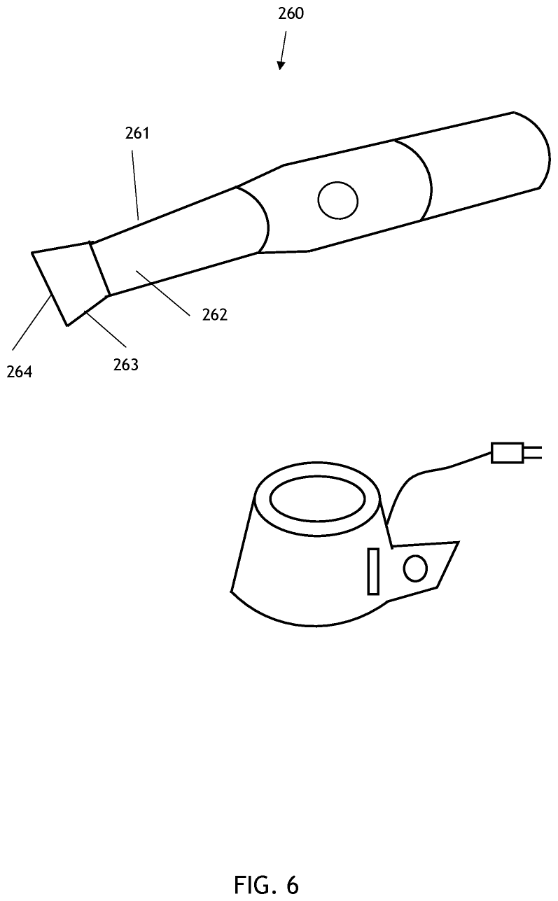

While the embodiments depicted in FIGS. 1-3 rely primarily on alternate heads to adapt the system to different purposes, a power control (107) is also provided to fine tune the system for given purposes. This control (107) is optional as the system can function for its purposes while relying entirely on the use of different heads. FIGS. 4-6 depict another schematic of invented system for curing light and therapeutic system using diode laser as light source, only without a power control switch. FIG. 4 is a curing light system and FIGS. 5 and 6 are therapeutic laser systems, respectively.

In FIG. 4, a curing light (200) is provided where (201) is the main body and with curing light emitter head (202) having a light exit (203). As with the previous embodiment, the direction of the exit (203) may be angled θ in a range 0 to 90 degrees respect to horizontal axis (dotted line in FIG. 2) of the curing head (202). As with the previous embodiment, the curing light (200) may be constructed of metal, plastic, composite, and any other durable materials and the curing head can be rotated around main body and be removable from the same. A single power button (204) may be provided with multiple functions. The button (204) can turn on and off the light for a fixed time, or cycle through frequency options. The button (204) may have multiple color backlights to indicate battery status and light emission status with different colors. The invented light can be powered by either AC/DC power or battery. If an AC/DC power is used, the power source can plug into the light directly and the power's plug can be used a main power switch and emergency stop. The unit may also be battery powered with a battery (205) that attaches the body (201) and that can be attached and detached easily to act as a main power switch and/or an emergency stop for the unit. A charging station (206) with an opening (207) for the battery or main body of the unit may also be provided. The charging station (206) is powered by a cord with wall plug (210). The battery (205) can be charged in a station with either contacts or wireless induction charging or by direct plugging in the unit to a power supply. As with the previous embodiment shown in FIGS. 1-3, system status may be measured and reported through a provided light intensity window (209) and an indicator (208).

A therapeutic cutting laser system (220) is achieved by changing the emitter head (221) of the system (FIG. 5). The therapeutic emitter head features a main body (222) can be made of metals, plastic, composite, and any other durable materials and features a cannular portion (223) through which an optical fiber (224) is extruded from the cannular portion. The cannular portion (223) can be made of metal or plastic and can be bendable to any angle as desired. The fiber (224) can be as small as 100 micrometers. As with the first system embodiment, the head (221) can utilize different configurations to deliver different sizes/shapes of the beam.

FIG. 6 depicts a large-beam therapeutic system (230). This embodiment is identical to the previous two, except that the head (231) emits a large beam for therapeutic purposes. As before, the main body (232) can be made of metals, plastic, composite, and any other durable materials. A cone (233) is located at the light exit (234) to allow passage of the broader beam. Beam size can be affected by the exit's size and shape.

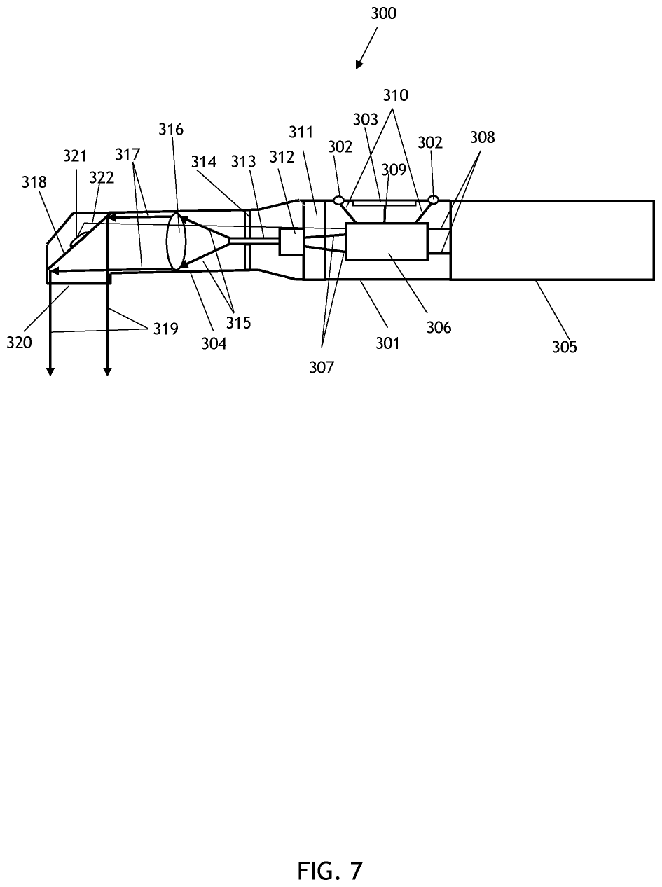

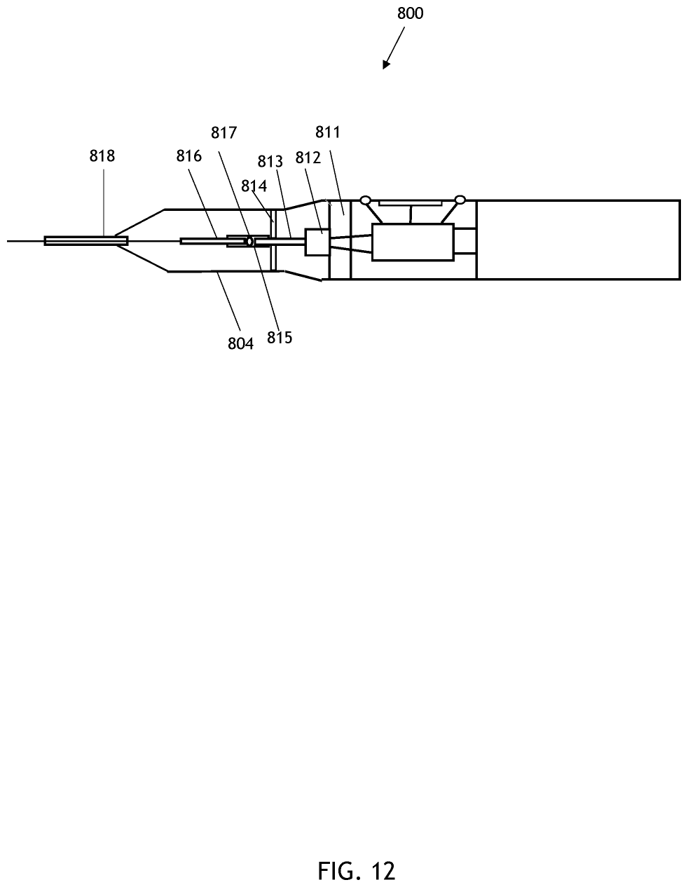

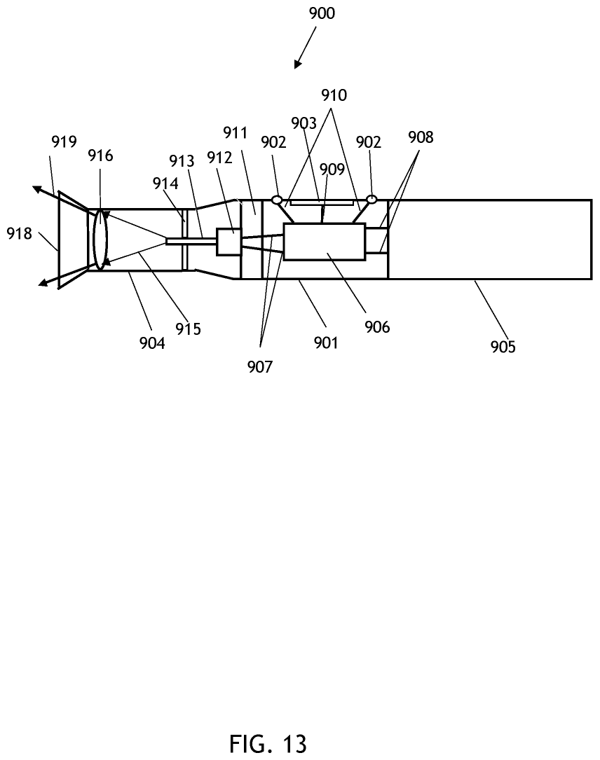

There are many potential designs for heads for different functions. FIGS. 7 through 11 depict various embodiments for curing light heads, while FIGS. 12 and 13 depict therapeutic heads. These designs are exemplary, and do not necessarily work with each other, but are shown to depict some of the many designs which may be utilized in the practice of this invention. In FIG. 7, one embodiment of the system handpiece (300) features a main handpiece housing (301) with control buttons (302) and a display (303). A head housing (304) is removable and exchangeable from handpiece body (301) while a battery or other power supply (305), such as an AC/DC power supply, is also provided. A control circuit (306) controls the light power output, laser operation control (including time), output power, pulse rate, battery status, and other features that are required for curing light and laser system operations. There are connections from control circuit to different components: (307) including connections (307) to laser module (312); connections (308) to a battery or AC/DC power supply (305); connection (209) to a display (303); and connections (310) to control button(s)(302). The laser module (312) is ideally mounted upon a heat sink (311). At this point, an optical system, which includes fiber (313), collimating lens (316) and reflector (318), converts the light emitted from laser module (312) into a collimated beam (319). A fiber (313) attached to laser module (312) initially collects emitted light and directs beam (315) into collimating lens (316) which then converts the beam into a collimated, parallel beam (317), as is required in curing operations. Fiber (313) may be terminated with ferrule or be free standing with cleaved interface on the fiber side. The length of the fiber (313) depends on the requirement of head (304). The size or diameter of the fiber can be ranged from 50 to 1000 μm. A holder (314) may be used to hold the fiber (313) into a position. The position of the lens from end of fiber depends on the focal length of the collimating lens (316) and the size of the parallel beam (317) will depend on diameter of the collimating lens (316). The parallel beam (317) travels to a reflector (318) which will turn the beam (317) as required for the geometry of the head (304). The depicted reflector (318) is positioned in a 45-degree angle in respect to lens (316) to turn the light beam to 90-degree direction to form a beam (319) to reach to wand exit (320). The position or angle of the reflector (318) can vary to conduct light in different directions and along different angles. The distance between lens and reflector depends on the requirement of head length. A photo detector (321) may be provided to measure the light intensity and feedback the signal through connection (322) to control circuit (306), which may then adjust the light intensity based on this feedback signal. All the components after the fiber holder (314) are in the head housing (304) and can be removed along with the head from handpiece body (301).

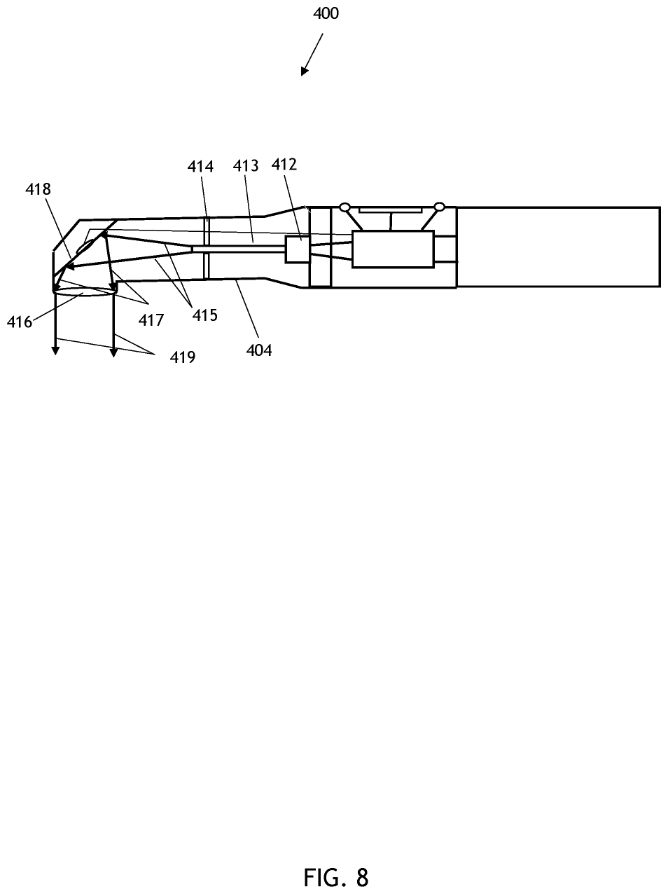

FIG. 8 depicts the same system as in FIG. 7, utilizing an alternate curing light head design (400). After laser module (412) emits a laser beam (415) through fiber (413), the beam (415) travels to a reflector (418) which directs beam (417) towards lens (416) positioned proximate the exit. Collimating lens (416) converts beam (417) into parallel beam (419) for use in curing applications. All the components after fiber holder (414) are in the head housing (404) and can be removed along with the head from the handpiece. The optical system which converts the light emitted from laser module (412) parallel beam (419) includes fiber (413), reflector (418), and collimating lens (416).

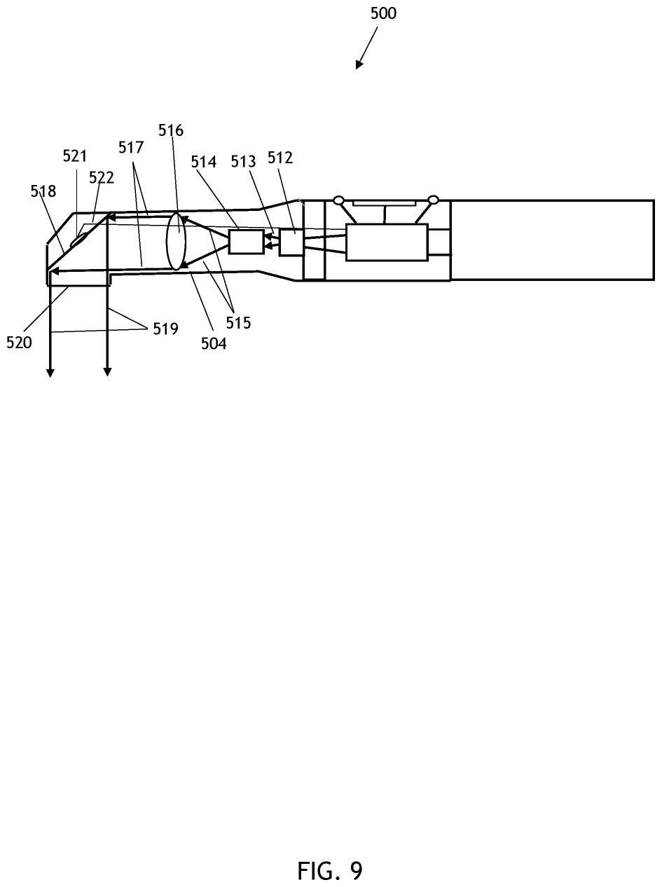

FIG. 9 also depicts the same system as in FIG. 7, utilizing an alternate curing light head design (500). In this embodiment laser module (512) emits a beam (513) into lens (514). It is common that the beam (513) to be an oval shape. Lens (514) may then focus the beam (513) to a point, then into a circular beam (515). There is a collimating lens (516) that converts the light beam (515) to a parallel beam (517). The position of the lenses relative to each other and laser module (312) will depend on their focal lengths and the size of the parallel beam will depend on diameter of the lens (516). It is possible for lenses (514) and (516) can be a single lens, depending on a design which will achieve a parallel beam from the emitted light directly from laser module (512). The parallel beam (517) travels to a reflector (518) which directs a reflected beam (519) to the wand exit (520). The components after laser module (512) will be in the head housing (504) and can be removed from the handpiece. The optical system which converts the light emitted from laser module (512) parallel beam (519) includes lenses (514) and (516), and reflector (518).

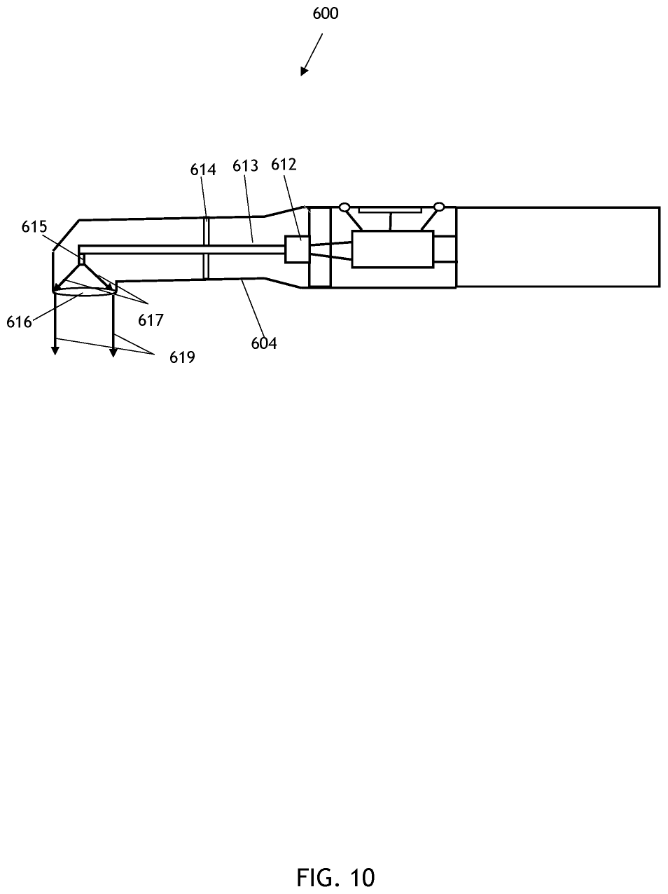

FIG. 10 also depicts the same system as in FIG. 7, utilizing another alternate curing light head design (600). In this embodiment, a fiber (613) may be attached to the laser module (612) and extends towards the end of head (604). It then makes a 90-degree turn (615) and points towards the light exit. A light beam emitted from the laser module (612) travels the fiber (613) and is emitted as beam (617). A collimating lens (616) situated at exit will convert the laser beam to a parallel beam (619). The direction of light exit in respect to horizontal axis of curing head is determined by the angle of fiber (615). The components after fiber holder (614) will be in the housing and can be removed from the handpiece while a holder (614) is provided to stabilize the length of fiber (613) extending from the laser module (612). The optical system which converts the light emitted from laser module (612) parallel beam (619) includes fiber components (613) and (615) and collimating lens (616).

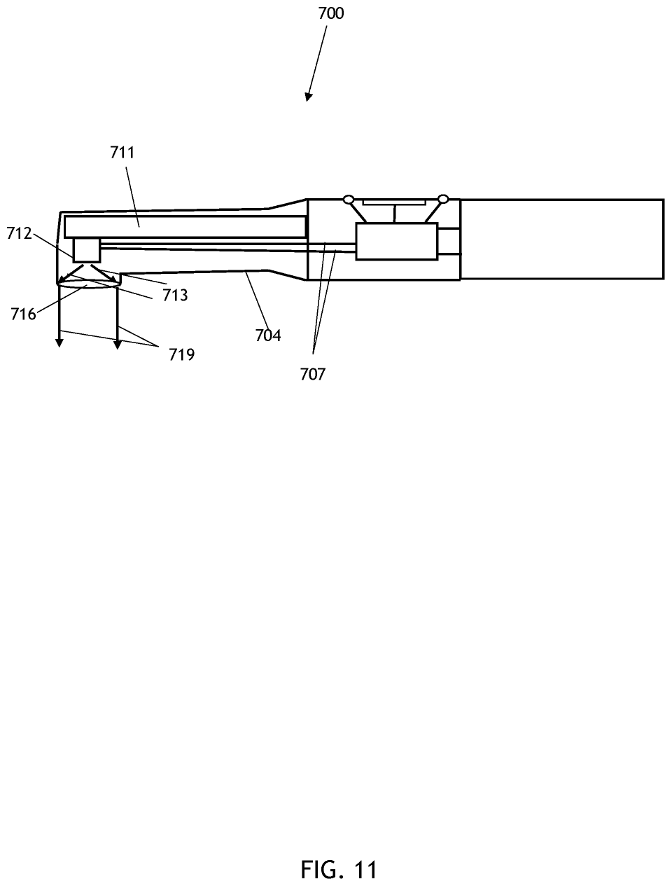

FIG. 11 also depicts the same system as in FIG. 7, utilizing another alternate curing light head design (700). In this embodiment, heat sink (711) is positioned inside the head housing (704) along most of its length. Module connections (707) likewise extend into the head housing (704). A laser module (712) is attached to the heat sink (711) and connections (707) and emits a beam (713). The beam travel to a collimating lens (716) which is at an exit of head housing. The lens (716) converts the laser beam to a parallel beam (719). The direction of light exit in respect to horizontal axis of curing head is determined by the position of laser module (712) and lens (716). The laser module (712), its connection, and lens (716) are part of housing (704) and can be removed from handpiece if needed. The optical system which converts the light emitted from laser module (712) parallel beam (719) includes collimating lens (716).

FIG. 12 also depicts the same system as in FIG. 7, utilizing a therapeutic laser head for use in surgery or other therapeutic applications (800). Laser module (812) is mounted upon heat sink (811) and has a fiber (813) attached thereto. The fiber (813) may be terminated with ferrule or be free standing with a cleaved interface on fiber side and the size or diameter of the fiber can be ranged from 50 to 1000 μm. A holder (814) 814 secures the fiber (813) into a position and a coupler (815) is provided to align fiber (813) from laser module (812) to fiber (816) in the head housing (804). The head fiber (816) can also be terminated with a ferrule or be free standing with a cleaved interface. The coupler (815) shall have a tight tolerance to align the two fibers and ensure the laser beam transmitted from fiber (813) to head fiber (816) has a minimal loss. Coupler (815) may feature an optional lens (817) between fiber (813) and head fiber (816). The lens (817) can couple the light between fibers to increase transmission efficiency. The head fiber (816) is further extended to outside of housing (804) through a bendable tubing (818) which may be used to bend head fiber (816) to any angle as desired by bending the tube (818). All the components after fiber holder (814) in the head housing (804) can be removed along with the head from the handpiece.

FIG. 12 also depicts the same system as in FIG. 7, utilizing a therapeutic laser head for use in surgery or other therapeutic applications (900). Some therapeutic applications only require a broad beam of light of a given frequency, this configuration (900) emits broad, non-cutting, non-collimated light. Laser module (912) is mounted upon heat sink (911) and has an attached fiber (913) extending therefrom. The fiber (913) may be terminated with ferrule or be free standing with a cleaved interface on fiber side. As in other embodiments, the size or diameter of the fiber can be ranged from 50 to 1000 μm. A holder (914) secures the fiber into a position. The fiber (913) provides a laser beam (915) in the housing (904) to a lens (916) which will enlarge and shape the beam with desired size and shape and guide the light (919) to conical exit (918). All the components after fiber holder (914) are in the head housing (904) and can be removed along with the head from the handpiece.

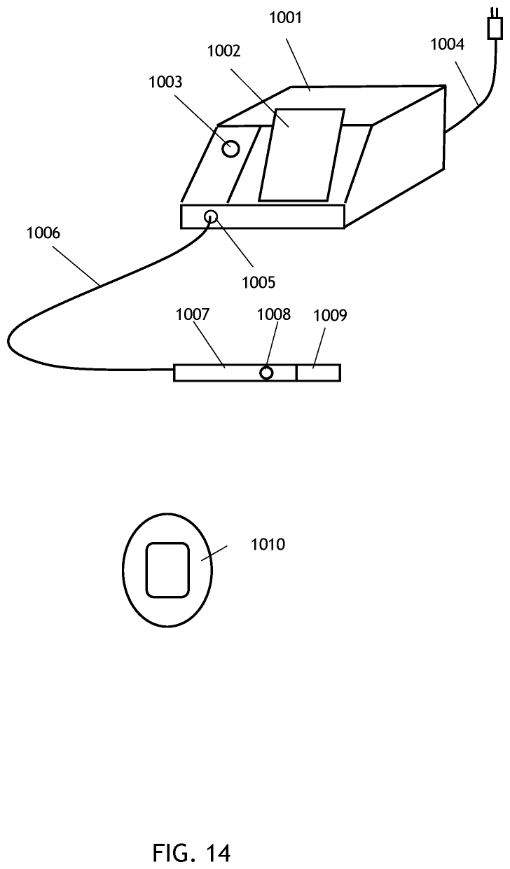

Any of the above heads may be utilized in a conventional operation unit, such as the desk top unit (1000) shown in FIG. 14. Desk top unit (1000) has a main body (1001) and a control display (1002) for system operations. The control display (1002) can be a touch pad, a touch screen, or removable module like iPad. Power is provided through power supply (1004) and the unit should have an emergency stop (1003). The system can utilize a rechargeable battery or AC/DC power input. Fiber (1005) and a control cable (1006) extend to a handpiece (1007) on which an attachment (1009) is attached. This handpiece attachment (1009) can be curing light tip, a fiber tip or a therapeutic tip as described above. Control switches may be located on the handpiece (1008) or through a footswitch, such as wireless footswitch (1010). The system can be controlled in either switch on hand piece (1008) or wireless foot switch (1010) while finer details may be controlled on the control display (1002).

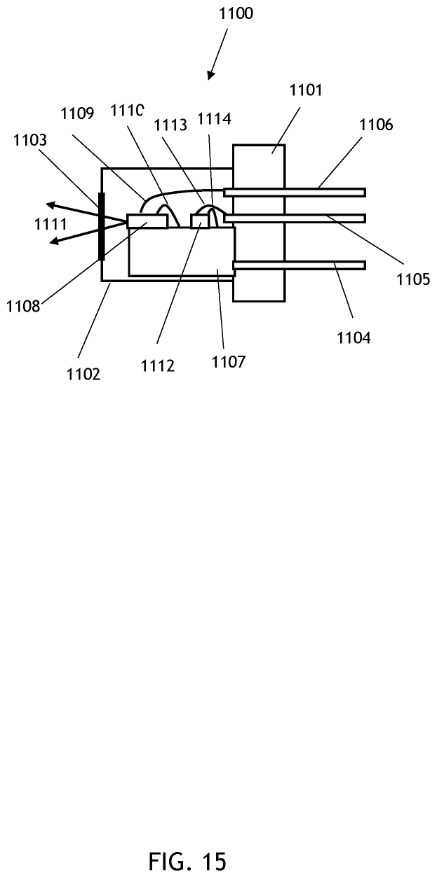

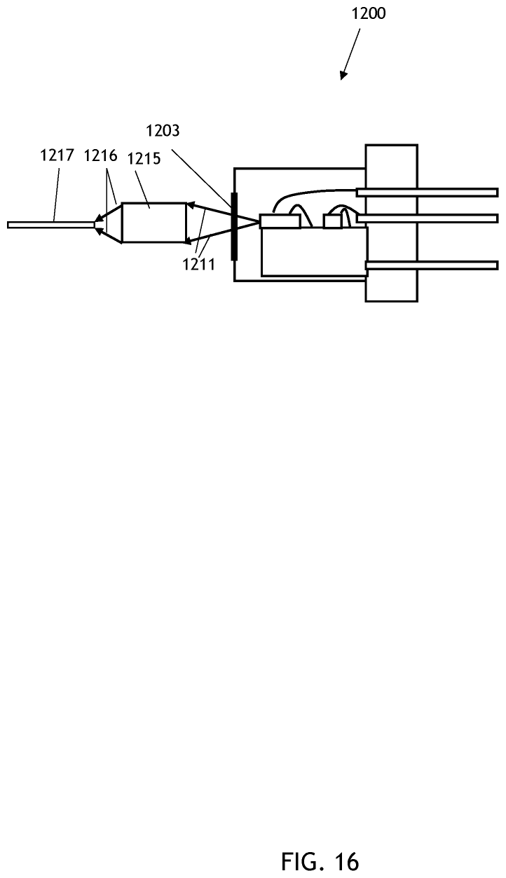

FIGS. 15 and 16 depict alternate embodiments for a laser module. In FIG. 15, laser module 1100 presents a base (1101) and casing (1102), where window (1103) in the casing (1102) allows emitted light to exit. The casing (1102) and base (1101) are generally made of metal or any heat conductive materials. There are electrodes for power input to the laser (1106) and detector (1105) chips inside laser module, where (1104) is a common electrode. Inside the casing, there is a heat sink (1107) attached to the base (1101). At least one laser chip (1108) is attached to heat sink (1107) and to common electrode (1104) and chip electrode (1106) through wires (1109) and (1110) respectively. The laser chip (1108) shall emit the light required for system operations. The laser chip (1108) can be a single chip or a chip array or multiple chips and be made of AlGaInN, GaInP, AlGaAs, or other compounds. The wavelength of light emitted by laser chip (1108) shall be that or those required by the system. For example, wavelengths 400 nm-480 nm can be used for bacterial reduction and curing. Wavelengths ranged around 650 nm can be used for pain therapy. Typical wavelengths for curing composites or adhesives can be in the range from 280 nm to 520 nm. Typical wavelengths for surgical and other therapeutic uses can be 650 nm, 780 nm, 810, 980 nm, 1160 nm and others. The laser module should be capable of emitting radiant energy at more than one discrete wavelength (about 50 nm apart). The beam emitting side of laser chip is aligned with window (803) and usually emits a divergent beam (1111). An optional photo detector (1112) may be attached to heat sink (1107) or at any position inside the casing (1102). The photo detector (1112) may be used to monitor the laser chip emitting power as feedback for controlling laser output. The photo detector (1112) can be connected to detector electrode (1105) and common electrode (1104) through wires (1113) and (1114) respectively. The purpose is photo detector (1112) is to measure the light from laser module (1100) and provide a feedback signal to the control circuit to control light beam emission of laser chip. An alternate laser module (1200) depicted in FIG. 16, features an optional lens structure proximate exit window (1203). Lens (1215) collects laser beam and converts it into a beam (1216) which will be focused to a point at end a fiber (1217). Thus, the laser light is incorporated to a fiber for further transportation in the fiber. The diameter if the fiber can be ranged from 50 to 1000 μm and can be terminated with or without a ferrule. If multiple chips are used in the laser module, then an optical system needs to be utilized to incorporate the laser beams from multiple chips into the fiber.

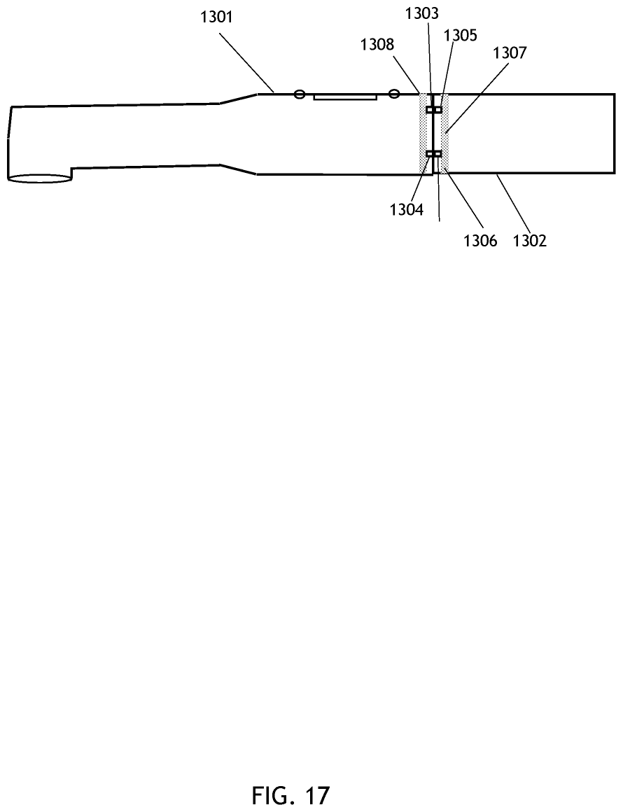

FIG. 17 illustrates one embodiment of battery attachment, which can be used as main power switch and emergency button for the laser units. Battery (1302) is attached to the handpiece of the light unit (1301). At end of handpiece (1301) are two electrical contacts (1303) and (1304). Battery (1302) also features two electrical contacts (1305) and (1306). The contacts in both bodies are mechanically aligned and contact each other when two bodies are attached. The attachment of two bodies may be facilitated using magnets, where at least one magnet (1307) is positioned in battery body and at least one other (1308) is positioned in the handpiece. In the practice, there may be magnets in either handpiece (1301) or battery body (1302) and the casing of the other is made of magnetically attractive materials like iron. The strength of magnets shall be selected to hold battery to main body when two bodies together and can also easily detached from handpiece.

Magnetic securement may also be utilized in securing the head to the handpiece as well. FIG. 14 illustrates one embodiment of head attachment for invented system where the handpiece of the curing light (1401) and the head (1402) are removable. At end of handpiece (1401) there are at least one electrical contact (1403). In the head body (1402), there corresponding electrical contacts (1404). The contacts in both bodies are mechanically aligned and contact each other when two bodies are attached. The contact in each body can be multiple pins to transfer different signals. The bodies may be secured with magnets, where at least one magnet (1405) is positioned in curing head body (1402) and at least one other (1406) is positioned in handpiece body (1401). As with the battery described above, there may be one magnet or set of magnets in either handpiece or the curing head body and the casing of the other may contain magnetically attractive materials like iron. The strength of magnets shall be selected to hold curing head to main body when two bodies together and can also easily detached from the handpiece.

Although the present invention has been described with reference to preferred embodiments, numerous modifications and variations can be made and still the result will come within the scope of the invention. No limitation with respect to the specific embodiments disclosed herein is intended or should be inferred.

1. A system combining therapeutic lasers and curing lights, the system comprising:
a powered radiant emission source and a collection fiber operably in communication therewith to channel radiant energy from said radiant emission source and direct the radiant energy towards an emitter head mounted upon a handpiece;
a plurality of exchangeable emitter heads, each emitter head selectable to a given purpose and said plurality of emitter heads including at least two emitter heads selected from the set of emitter heads consisting of:
a head that receives radiant energy from the collection fiber and directs said radiant energy into a collimating lens, which then collimates the radiant energy and emits it as a curing light;
a head that receives radiant energy from the collection fiber and directs said radiant energy into a therapeutic fiber, which then concentrates the radiant energy into a focused, cutting laser; and,
a head that emits the radiant energy as a non-collimated therapeutic light;
wherein one emitter head is selected to transform and emit radiant energy according to a given procedure and attached to the system for use.

2. The system of claim 1, the radiant emission source being a laser module capable of emitting radiant energy at a plurality of discrete frequencies and the system further comprises a control system to select one discrete frequency to emit at a time.

3. The system of claim 1, the means to channel radiant energy including at least one optical fiber.

4. The system of claim 1, the means to channel radiant energy including at least one lens.

5. The system of claim 1, the means to channel radiant energy including at least one reflector.

6. The system of claim 1, each emitter head in the set of emitter heads being selectively magnetically secured to the handpiece.

7. A curing light comprising:
a handpiece containing a laser module and a means for powering the laser module, and
a curing light head which further comprises an optical system for converting light emitted from the laser module into a beam of collimated light;
the optical system further comprising a collimating lens;
wherein light emitted from the laser module is directed towards the optical system where it interacts with the collimating lens to be converted into a collimated beam.

8. The curing light of claim 7, the optical system further comprising an optical fiber which collects light emitted from the laser module and directs said light towards the collimating lens.

9. The curing light of claim 7, further comprising a second, directive, lens.

10. The curing light of claim 7, further comprising a reflector.

11. The curing light of claim 7, further comprising a detector to detect light intensity.
